(19) United States
(12) Patent Application Publication
(10) Pub. No.: US 2025/0325361 A1
(43) Pub. Date: Oct. 23, 2025

(54) METHODS, SYSTEMS, AND DEVICES FOR EMBOLIC PROTECTION

(71) Applicant: Baleen Medical, LLC., Palo Alto, CA (US)

(72) Inventors: Sebastian KHAIRKHAHAN, Palo Alto, CA (US); Alex KHAIRKHAHAN, Palo Alto, CA (US)

(21) Appl. No.: 19/195,431

(22) Filed: Apr. 30, 2025

Related U.S. Application Data
(63) Continuation of application No. PCT/US2023/079265, filed on Nov. 9, 2023.
(60) Provisional application No. 63/424,121, filed on Nov. 9, 2022.

(51) Int. Cl. A61F 2/01

(52) U.S. Cl. CPC A61F 2/013; A61F 2/0105; A61F 2/011

(57) ABSTRACT

Embodiments of the present disclosure are directed to methods, systems and devices for embolic protection, and more specifically, to methods, systems, and devices for embolic protection for surgical systems and methods including, for example, delivery/implanting systems as well as methods for delivering or implanting prosthetic heart valves into the heart, or performing a cardiac or blood vessel procedure, where capturing or otherwise trapping emboli dislodged or created during the procedure is necessary so as to prevent complications associated therewith (e.g., strokes).

### CROSS-REFERENCE TO RELATED APPLICATIONS

This application is a continuation of PCT/US2023/079265, filed Nov. 9, 2023, which claims benefit of and priority to U.S. provisional patent application No. 63/424, 121, filed Nov. 9, 2022, and entitled, “METHODS, SYSTEMS, AND DEVICES FOR EMBOLIC PROTECTION.” The subject disclosure is also related to PCT publication no. W02020/102679. Each of the preceding disclosures are herein incorporated by reference in its entirety.

### BACKGROUND OF THE DISCLOSURE

In the past decade, major developments have taken place in catheter-based treatments for structural heart diseases such Transcatheter Aortic Valve Replacement (TAVR) and Transcatheter Mitral Valve replacement (TMVR). Cerebral embolism is a known complication of such procedures where embolic particles which may include thrombus, atheroma, and lipids may dislodge during the implantation procedure, enter the blood stream embolizing in the brain and other vital organs. Cerebra embolism could result in serious neurological deficits, stroke, and even death. Furthermore, embolism in vital organs such as kidneys could severely compromise the function of these organs resulting in hospitalization, diminished quality of life and in some cases death. Therefore, preventing the embolism in the brain and other vital organs could hugely benefit the patient by improving the outcome of these procedures.

### SUMMARY OF THE DISCLOSURE

Embodiments of the present disclosure are directed to methods, systems and devices for embolic protection, and more specifically, to methods, systems, and devices for embolic protection for surgical systems and methods including, for example, delivery/implanting systems as well as methods for delivering or implanting prosthetic heart valves into the heart, or performing a cardiac or blood vessel procedure, where capturing or otherwise trapping emboli dislodged or created during the procedure is necessary so as to prevent complications associated therewith (e.g., strokes).

It is worth noting that the systems, apparatuses and devises disclosed herein, may, in some embodiments, be referred to as an embolic protection apparatus, an embolic protection assembly, an embolic protection system, an embolic protection device, such terms/phrases can be used interchangeably here throughout, and can be referred to as the acronym “EPA” throughout.

In some embodiments of the present disclosure, an embolic protection apparatus (EPA), which can also be considered a system or a device, includes an expandable filter arranged on a distal end of the apparatus which includes a plurality of pores sized to allow the flow of the blood with limited interruption and capture of emboli greater than the pore size. The apparatus can also include one or more of the following, and preferably, in some embodiments, a plurality of (and in some embodiments, all of) an expandable shaft for at least housing the expandable filter prior to deployment of the filter, a filter control wire, a control handle including a filter deployment knob configured to move along a filter deployment helix, a filter eyelet, and a helix gasket. In some embodiments, one or more of the preceding elements may be included with the EPA.

In some embodiments of the present disclosure, an embolic protection apparatus (EPA) which includes an expandable filter arranged on a distal end of the apparatus and including a plurality of pores sized to allow the flow of the blood with limited interruption and capture of emboli greater than the pore size, an expandable shaft for at least housing the expandable filter prior to deployment of the filter, a filter control wire, and a control handle. In some embodiments, the EPA may also include a filter deployment knob to control filter deployment, which in some embodiments, may be configured to engage and/or move along a filter deployment helix (this deployment know and/or helix may, in some embodiments, be optional).

Such embodiments (as well as other embodiments disclosed herein, including any system, apparatus, device, or method embodiments summarized below, including those in paragraphs [0008]-[0012], or detailed in the Detailed Description which follows, may further include one and/or another of the following features, structure, functionality, steps, or clarifications (and in some embodiments, a plurality of, and in some embodiments, a majority of, and in some embodiments, substantially all of, and in some embodiments, all of), yielding yet further embodiments of the present disclosure:
the expandable shaft may include a tapered distal end;
an eyelet, which may be located on a portion of the expandable filter and/or configured to allow passage of at least one of a guide wire, dilator and one or more therapeutic devices;
the eyelet may be configured to be flexible, where the flexibility of the eyelet can be configured so as to accommodate passage therethrough of elements or devices of varying diameters.
the expandable filter is connected to the filter deployment helix by at least one connector wire or cable;
a/the connector wire or cable is comprised of at least one of stainless steel and Nitinol;
a deployment helix, which may be engaged with a filter deployment knob;
upon rotation of the filter deployment knob in a first direction, the filter is directed out of a distal end of the expandable shaft;
the control handle includes a port configured to provide at least one of flushing and irrigating a component of the apparatus, where, in some embodiments, the component is selected from the group consisting of: the expandable shaft, guide wire, the connector wire, and the control handle;
a/the gasket is affixed to the control handle and is configured to prevent leakage from the apparatus during use;
a/the gasket prevents leakage from at least one of insertion and removal of therapeutic devices from the apparatus;
a proximal end of the filter is connected to a distal end of a wire or cable (such structure may be that of the above disclosed connector wire or cable);
a proximal end of the wire or cable is connected to the filter deployment helix for enabling linear movement of the expandable filter inside the expandable shaft;
the wire or cable is comprised of stainless steel or nitinol;
a gasket is mounted on a distal end of the filter control helix and is configured to seal the proximal end of the expandable shaft;
a first dilator;
a gasket which includes an opening for another element, including, for example, a distal end of a dilator;
the first dilator comprises, depending upon the embodiment(s), at least one of, at least a plurality of, at least a majority of, substantially all of, and all of:
   a dilator nosecone;
   a dilator outer shaft;
   a dilator inner shaft;
   a dilator control handle;
   a nosecone control knob;
   a dilator helix; and
   a flush port;
a proximal end of at least one of the inner shaft and the outer shaft is attached to the dilator helix;
a distal end of at least one of the inner shaft and the outer shaft is attached to the dilator nosecone;
a proximal end of the outer shaft is attached to the control handle;
a distal end of the outer shaft is tapered;
a distal end of the outer shaft is configured to transition to at least one of the nosecone and the inner shaft;
a dilator helix is engaged with a nosecone control knob, where, in some embodiments, rotation of the nosecone control knob in a first direction can causes the nosecone to move in a distal direction;
as a result of the nosecone being moved in the distal direction, the nosecone can be disconnected or undocked from a distal end of outer shaft;
a dilator helix is engaged with a nosecone control knob, where, in some embodiments, rotation of the nosecone control knob in a second direction causes the nosecone to move in a proximal direction;
as a result of the nosecone moving in a proximal direction, the nosecone is connected or docked with a distal end of the outer shaft;
a radiopaque marker, which may:
   function as stop for cessation of movement of the nosecone; and/or
   be configured to enable verification of at least one of the movement of the nosecone and undocking and docking of the nosecone with the distal end of the outer shaft under fluoroscopy;
the inner shaft includes at least one lumen, where the at least one lumen can be configured to receive a guidewire;
a distal end of at least one of the inner shaft and the outer shaft is comprised of a flexible material;
a distal end of the expandable shaft is tapered to transition to the distal end of the first dilator, where:
   the tapered distal end of the shaft can be comprised of a material capable of being fractured or broken under an outward radial force; and/or
   the tapered distal end of the expandable shaft includes longitudinal scoring;
   a/the scoring is configured to enable controlled fracturing or breaking of the distal end;
a second dilator, where the second dilator in some embodiments includes a diameter greater than a diameter of a/the first dilator; and
the second dilator comprises, depending upon the embodiment(s), at least one of, at least a plurality of, at least a majority of, substantially all of, and all of:
   a dilator nosecone;
   a dilator outer shaft;
   a dilator inner shaft;
   a dilator control handle;
   a nosecone control knob;
   a dilator helix; and
   a flush port.

In some embodiments, an embolic protection apparatus (EPA) is provided and includes an expandable filter arranged on a distal end of the apparatus, the filter can include a plurality of pores sized to allow the flow of the blood with limited interruption and capture of emboli greater than the pore size. The apparatus can also include an expandable shaft for at least housing the expandable filter prior to deployment of the filter, a filter control wire, a control handle including a filter deployment knob configured to move along a filter deployment helix, and a dilator including a dilator nosecone, a dilator outer shaft, a dilator inner shaft, a dilator control handle, a nosecone control knob, a dilator helix.

In some embodiments, an embolic protection apparatus (EPA) is provided and includes an expandable filter arranged on a distal end of the apparatus which can include a plurality of pores sized to allow the flow of the blood with limited interruption and capture of emboli greater than the pore size, an expandable shaft for at least housing the expandable filter prior to deployment of the filter, a filter control wire, a control handle including a filter deployment knob configured to move along a filter deployment helix, a first dilator including a dilator nosecone, and a dilator outer shaft, and a second dilator including a second dilator nosecone and a second dilator outer shaft.

In some embodiments, an embolic protection method including optionally providing an embolic protection apparatus (EPA) according to any one or more of the EPA embodiments disclosed herein, inserting a guide wire into an anatomy of a patient, advancing the expandable shaft of the EPA containing the expandable filter of the EPA over the guide wire, undocking the nosecone from a distal end of the shaft of the EPA via rotation of a dilator control knob in a corresponding first direction, advancing the expandable filter from the distal end of the EPA and dilator shaft via rotation of the filter deployment control knob of the EPA in a corresponding first direction, wherein a length of the expandable filter is adjusted via rotation of the filter deployment control knob in a corresponding first and/or second direction, positioning the expandable filter in the intended treatment site via at least one of rotation of the filter deployment control knob in the corresponding first and/or second direction, and movement of the expandable shaft, after completion of embolic protection: rotating the filter deployment control knob in the corresponding second direction so as to retract the expandable filter within the shaft; and rotating the dilator control knob in a corresponding second direction so as to dock a distal end of the dilator control knob with the distal end of the dilator shaft. The method can further include removing the EPA from the anatomy.

In some embodiments, an embolic protection method is provided, which can optionally include providing an embolic protection apparatus (EPA) according to any one or more of the disclosed EPA embodiments. Accordingly, the method may further include inserting the distal end of the expandable shaft into the vasculature of a patient, the expandable shaft having therein the dilator such that the nosecone of the dilator is the most distal element of the EPA, positioning the distal end of the EPA such that the expandable filter in the undeployed state is positioned at or adjacent an intended treatment site, removing the dilator from the EPA, optionally, adjusting the location of the filter in an undeployed state, if necessary via movement of the EPA along vasculature, deploying the filter by via rotation of the filter deployment knob, wherein a length of the deployed filter can be adjusted via rotation of the filter deployment knob, inserting the second dilator, the second dilator configured to expand the expandable shaft and open the tapered distal end of the expandable shaft, optionally delivering a treatment device into the anatomy of the patient, the delivery occurring through the expanded filter, withdrawing or recapturing the expandable filter by rotation of the filter control knob in a second direction, and withdrawing the EPA from the patient.

An embolic protection apparatus comprising a dilator including a shaft, a distal tapered tip, a lumen for receiving a guidewire, and a luer-lock connector arranged at a proximal end thereof.

More embodiments are possible by combining embodiments (and one and/or another of the components, features, structure, functions, functionality, steps, and/or clarifications) disclosed in related to PCT publication no. W02020/102679, as previously incorporated in the present disclosure (see page 1, intro paragraph).

These and other embodiments, components, materials, steps, and advantages and objects thereof will become even more apparent with reference to the detailed description which follows, and reference to the associated figures, a brief description of which is provided below.

### BRIEF DESCRIPTION OF THE FIGURES

FIG. 1 is a side view of an expandable shaft and other components of an embolic protection apparatus (EPA), including an expandable filter (in an undeployed state) which is carried by the expandable shaft, according to some embodiments of the present disclosure;

FIG. 2 is a side view of the expandable shaft and other components of an EPA, including the expandable filter in a deployed state, according to some embodiments of the present disclosure;

FIG. 3 is a side view of the expandable shaft and other components of an EPA, including the expandable filter in a deployed state, as well as illustrating a guide wire used with the apparatus/system, according to some embodiments of the present disclosure;

FIG. 4 is a side view of a dilator for an EPA, according to some embodiments of the present disclosure;

FIG. 5 is a side view of the dilator of FIG. 4 for the embolic protection apparatus, illustrating a dilator nosecone undocked from a shaft of the dilator, according to some embodiments of the present disclosure;

FIG. 6 is a side view of an EPA assembly, according to some embodiments, illustrating the assembled apparatus;

FIG. 7 is another side view of the EPA assembly of FIG. 6, illustrating the nosecone of the dilator being uncoupled/undocked from the distal end of the dilator shaft, according to some embodiments;

FIG. 8 is another side view of the EPA assembly of FIG. 6, illustrating the nosecone of the dilator being uncoupled/undocked from the distal end of the dilator shaft, as well as the expandable filter being in a deployed state, according to some embodiments;

FIG. 9 is another side view of the EPA assembly of FIG. 6, which again, illustrates the nosecone of the dilator being uncoupled/undocked from the distal end of the dilator shaft, as well as the expandable filter being in a deployed state and the tapered end of the dilator at least partially within the nosecone, according to some embodiments;

FIG. 10 is a side view of the expandable shaft of the EPA assembly of FIG. 6, and the expandable filter in a deployed state after the dilator has been removed, according to some embodiments;

FIG. 11 is a side view of another EPA assembly of FIG. 6, according to some embodiments, which includes an expandable shaft with an expandable filter therein, as well as a dilator;

FIG. 12 is a side view of a dilator of the EPA assembly of FIG. 11, according to some embodiments of the disclosure;

FIG. 13 is a side view of a dilator of the EPA assembly of FIG. 11, illustrating movement of the dilator/hub (arrow) for removing the dilator (for example), according to some embodiments of the disclosure;

FIG. 14 is a side view of the expandable shaft of the EPA assembly of FIG. 11, according to some embodiments of the disclosure, illustrating the assembly without a dilator;

FIG. 15 is a side view of the expandable shaft of the EPA assembly of FIG. 11, according to some embodiments of the disclosure, illustrating the assembly without a dilator, and the expandable filter deployed;

FIG. 16 is a side view of the expandable shaft of the EPA assembly of FIG. 11, according to some embodiments of the disclosure, illustrating the assembly with a second dilator, and the expandable filter deployed; and

FIG. 17 is a side view of the expandable shaft of the EPA assembly of FIG. 11, according to some embodiments of the disclosure, as well as an additional dilator (separated), illustrating the assembly without the second dilator, and the expandable filter deployed.

### DETAILED DESCRIPTION

One of skill in the art will appreciate that the illustrations provided for in the figures illustrate at least some of the embodiments of the present disclosure, and for convenience, are shown such that elements are transparent so as to enable one of skill in the art to determine at least one of arrangement, positioning, and functionality of the structure of the illustrated embodiments. It will be appreciated that such elements in commercialization can be transparent, or opaque, or a combination thereof (e.g., shafts, housings, helixes, etc.).

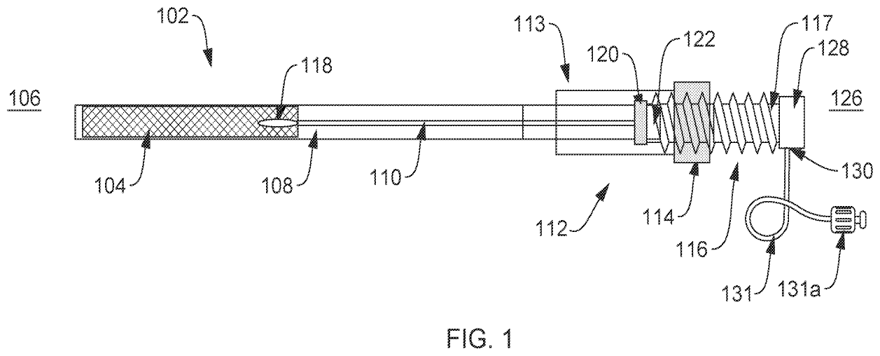

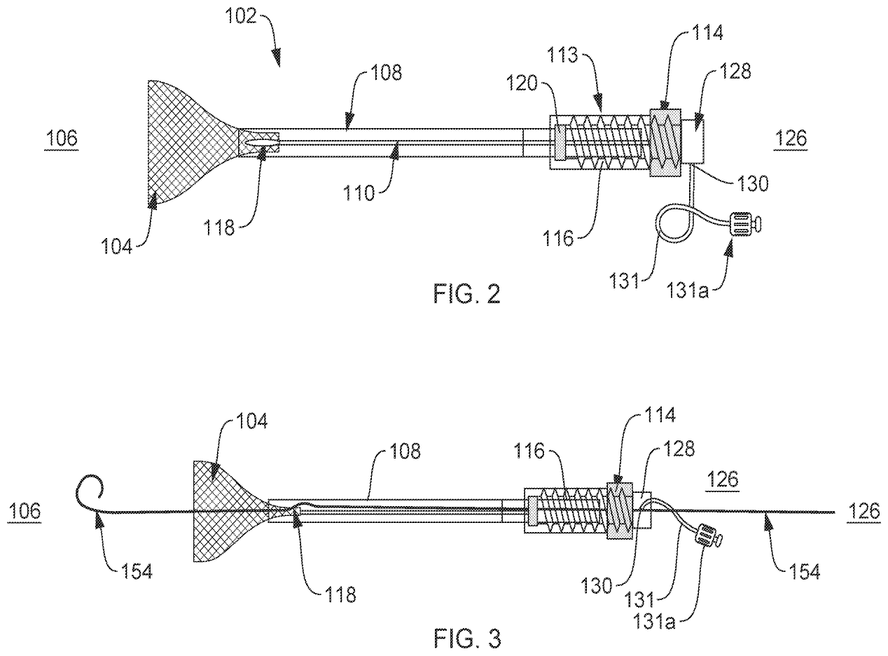

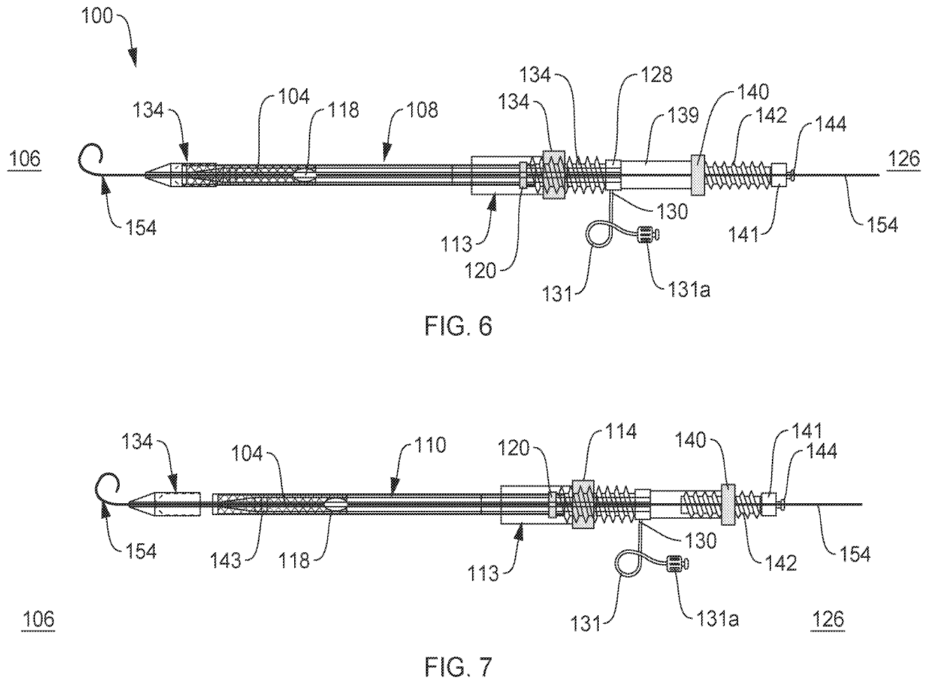

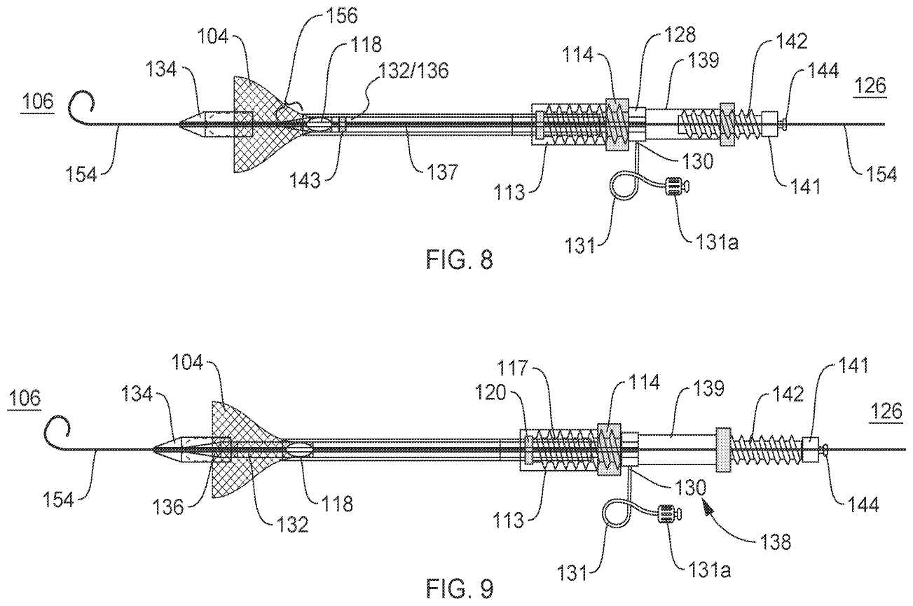

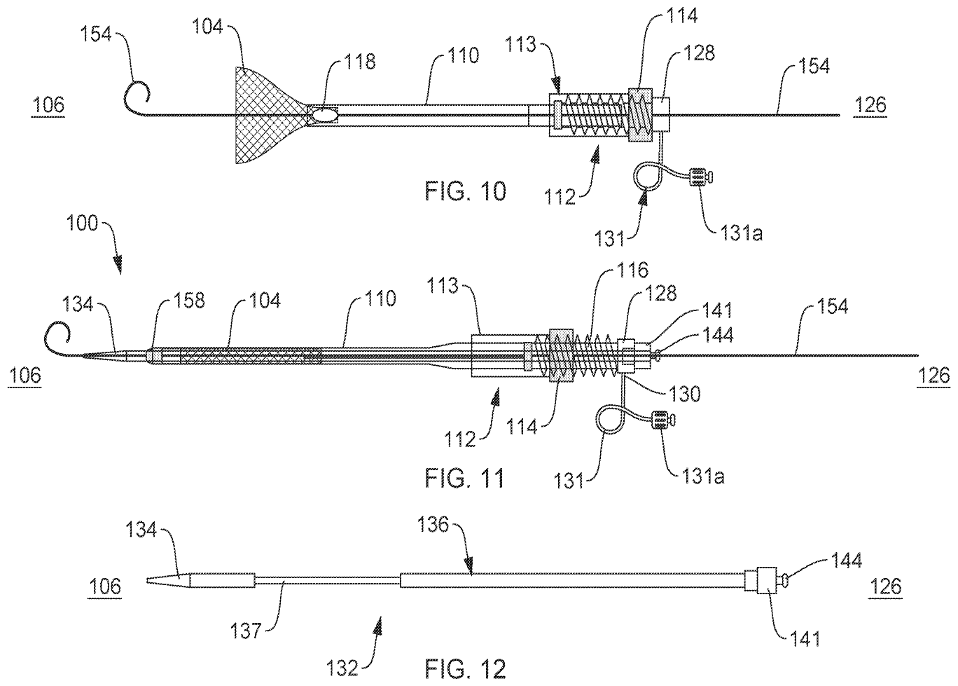

FIGS. 1-10 illustrate various embodiments according to the present disclosure. As shown, a first portion 102 includes an expandable filter 104 arranged on a distal end 106 of the first portion 102 (and the EPA in general) and includes a plurality of pores sized to allow flow of blood therethrough with limited interruption, and also sized to capture emboli contained within the blood greater than the pore size. In some embodiments, the size of the pores (or can be referred to as “holes”) can be from 80-300 microns, and in some embodiments, 120 microns or approximate thereto (and ranges between any of the foregoing).

First portion 102 also includes an expandable shaft 108 for at least housing the expandable filter 104 (e.g., prior to deployment of the filter). The first portion can further include a filter control wire 110, a control handle 112 including a filter deployment knob 114 configured to move along a filter deployment helix 116 (FDH). The FDH 116, in some embodiments, includes a spiral screw thread which threads with a corresponding internal screw thread (not shown) of the filter deployment knob 114. Thus, in some embodiments, the FDH 116 is engaged with the filter deployment knob. As shown in FIG. 1, the expandable filter is in an undeployed state within the distal end of the expandable shaft 108. In some embodiments, the distal end of the expandable shaft may be tapered (e.g., cone like in appearance, see similar features shown in FIG. 11, for example). Accordingly, in some embodiments, after the filter has been initially deployed; which due to the configuration and/or material of the filter, itself expands (in some embodiments, manual expansion of a filter may be performed, which may include, in some embodiments, methods familiar to those of skill in the art). With an expandable shaft with a tapered end, after filter deployment, the tapered end may be moved by the operator (either via rotation of the filter deployment knob or movement of the expandable shaft distally by the operator for example), so that the entirety of the interior diameter of the expandable shaft may be utilized for delivery of items (e.g., prosthetic valves, tools, and the like). This step can be used in one and/or another of various system, apparatus and method embodiments disclosed herein.

The expandable shaft can further include a filter eyelet 118, which can be located near the distal end 106, preferably spaced apart of the physical end of the expandable shaft 108, a filter eyelet 118, and a helix gasket 120. In some embodiments, the filter eyelet 118 can be configured to allow passage of at least one of a guide wire, dilator and one or more therapeutic devices, and in some embodiments, any and all of the preceding components. The eyelet can be configured to provide a passage for at least one of a guide wire, a dilator, and one or more therapeutic devices, through the inner surface of the filter, and thus, in some embodiments, the eyelet is configured to be flexible, which can be accomplished via use of a flexible material and/or shape/size of the eyelet 118. Accordingly, the flexibility of the eyelet 118 according to some embodiments is configured so as to accommodate passage therethrough of elements or devices of varying diameters.

In some embodiments, the control handle 112 can include a housing 113 that receives the expandable shaft 108 as well at its distal end, and which includes a proximal portion 126 with a hub 128, upon which the FDH 116 projects in the proximal direction. Accordingly, the proximal end 117 of the FDH includes an opening 1 for receiving a dilator (embodiments of which are detailed below) of the EPA. Reference no. 126 also refers to the proximal end of the EPA and components thereof in general.

The hub 128 includes a filtering/flushing port 130, which can include a tube 131 connected thereto; the free end of the tube 132 can include a connector 134 for connection to a fluid (e.g., flushing fluid, saline, and the like) source, where such connector may be a luer-lock connector 131a.

In some embodiments, the expandable filter 104 can be connected to the filter deployment helix 116 via at least one connector wire or cable 124. The connector wire or cable can be comprised of at least one of stainless steel and Nitinol metal. Accordingly, upon rotation of the filter deployment knob in a first direction, the filter is moved linearly out of the distal end 106 of the expandable shaft 108 (and EPA in general), and retracked by rotation of the filter deployment know in a second direction (e.g., opposite to the first direction), so as to be recaptured by the distal end of the expandable shaft 108 (for example) via linear movement. Thus, in some embodiments, a proximal end of the expandable filter 104 can be connected to a distal end of the connector wire or cable.

The control handle, in some embodiments, can include a port configured to provide at least one of flushing and irrigating a component of the apparatus, which, in some embodiments, can include any and all of the expandable shaft 108, a guide wire, the connector wire 124, and at least portion of the control handle 112.

In some embodiments, the gasket is affixed to the control handle 112 and is configured to prevent leakage from the apparatus during use. As shown in the figures, the gasket 120 can be within the control handle 112. Thus, the gasket 120 is sized to as to be seal at least one of the expandable shaft, the housing 113, and FDH 116. In some embodiments, and as shown in FIGS. 1-11, the helix gasket 120 is arranged on the distal end of the FDH 116, and thus, can at least seal the proximal end of the expandable shaft 108, as well as, in some embodiments, prevent leakage from at least one of insertion and removal of therapeutic devices from the apparatus.

In some embodiments, the EPA can also include a dilator which can be received within the expandable shaft 108 (although in some embodiments, it can be configured that the dilator receives the expandable shaft 108, and in such cases, the gasket 120 can be configured to accommodate and seal the dilator). In some embodiments, the dilator can be slidingly received by the proximal end of the expandable shaft.

In some embodiments, the dilator can include at least one of, in some embodiments, a plurality of, in some embodiments, a majority of, in some embodiments, substantially all of, and in some embodiments, all of a dilator nosecone 134, a dilator outer shaft 136, a dilator inner shaft 137, a dilator control handle 138 (which can include a dilator control handle housing 139), a nosecone control knob 140, a dilator helix 142, and a flush port 144. It is worth noting that the dilator shaft can be correspond to an outer (dilator) shaft, according to some embodiments. To this end, in some embodiments, a dilator inner shaft 137 is included as well for, in some embodiments, housing the guidewire 154. In some embodiments, the interior of the nosecone can be sized and shaped not only to connect/dock with the distal end of the dilator and/or expandable shaft, but also (in some embodiments) to receive a tapered distal end of the expandable shaft. In some embodiments, the distal end be spaced from the proximal end (or a distal end of the control handle portion, about 10-30 cm. The inner and outer shafts in some embodiments are flexible to allow for bending thereof due to the tortuosity of the vascular system of a patient.

In some embodiments, a proximal end 146 of the dilator outer shaft 136 is attached to the dilator helix 142, and a distal end 148 of the dilator outer shaft 136 can be attached to a proximal end 148 of the dilator nosecone 134. In some embodiments, a proximal end of the dilator outer shaft 136 can be attached to the dilator control handle 138. The distal end of the dilator control handle housing 139 can be connected to the dilator outer shaft 136 (and in some embodiments, the dilator inner shaft 137), and a proximal end of the housing 139 is affixed to the dilator helix 142 (i.e., a distal end of the dilator helix). In some embodiments, the dilator inner shaft 137 includes at least one lumen which can be configured to receive a guidewire 154.

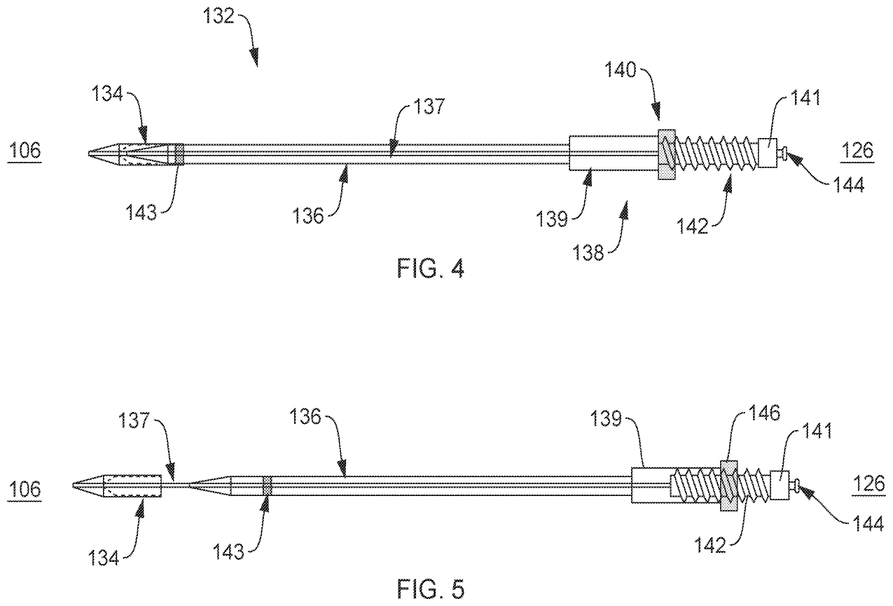

In some embodiments, the distal end of the outer shaft may be tapered. In such embodiments, such a tapered end (ref. no. 156, see FIGS. 4-5, 8-9, for example), can be received by an interior of the nosecone 134 that is sized and shaped to do so (according to some embodiments).

In some embodiments, a distal end of the dilator outer shaft (e.g., outer shaft) can be tapered as shown in FIGS. 4-5, such that, for example, the outer diameter of the outer shaft 136 shaft is configured to transition to a diameter of, for example, the inner shaft 137 (as well as, in some embodiments, or alternatively, taper to correspond to an internal taper of the nosecone 134). It is worth noting that, relative to the expandable shaft 108. In some embodiments, the distal end of the expandable shaft 136 is tapered to transition to the distal end of the dilator (and/or dilator nosecone 134).

Similar to the filter deployment helix 116, the dilator helix 143, in some embodiments, can include a spiral screw thread which threads with a corresponding internal screw thread of the nosecone control knob 140. Thus, in some embodiments, the dilator helix 142 is engaged with the nosecone control knob 140. Thus, rotation of the nosecone control knob 140 in a first direction causes the dilator nosecone 134 to move in a distal direction (so as to “de-dock” from the distal end of the outer shaft), and rotation of the nosecone control knob 140 in second direction (e.g., opposite to the first direction, clockwise/counterclockwise), causes the dilator nosecone 134 to move in a proximal direction (which can be to “dock” with the distal end of the outer shatter 136).

In some embodiments of the present disclosure, one or more components of the EPA can include a radiopaque marker 143, which, in some embodiments, can be arranged or positioned to function as stop for the cessation of movement of the nosecone (and example of this functionality is illustrated in FIG. 4). In many embodiments, the radiopaque marker is configured to enable verification of at least one of the movement of the nosecone 134 and/or the undocking and/or docking of the nosecone 134 with the distal end of the outer shaft 136 under fluoroscopy. One or more radiopaque markers can be included on one and/or another components of the EPA.

Any shaft of the EPA, according to some embodiments of the disclosure, and in particular (according to some embodiments) the distal ends of at least one of the inner shaft 137 and the outer shaft 136, can be comprised of a flexible material.

In some embodiments of the present disclosure, a distal tip of the expandable shaft is formed to taper to a lumen. For example, an initial outer diameter of the expandable shaft and the tapered tip can be from approximately 2-10 mm (for example), and ranges therebetween (according to some embodiments), and in some embodiments, about 4-5 mm.

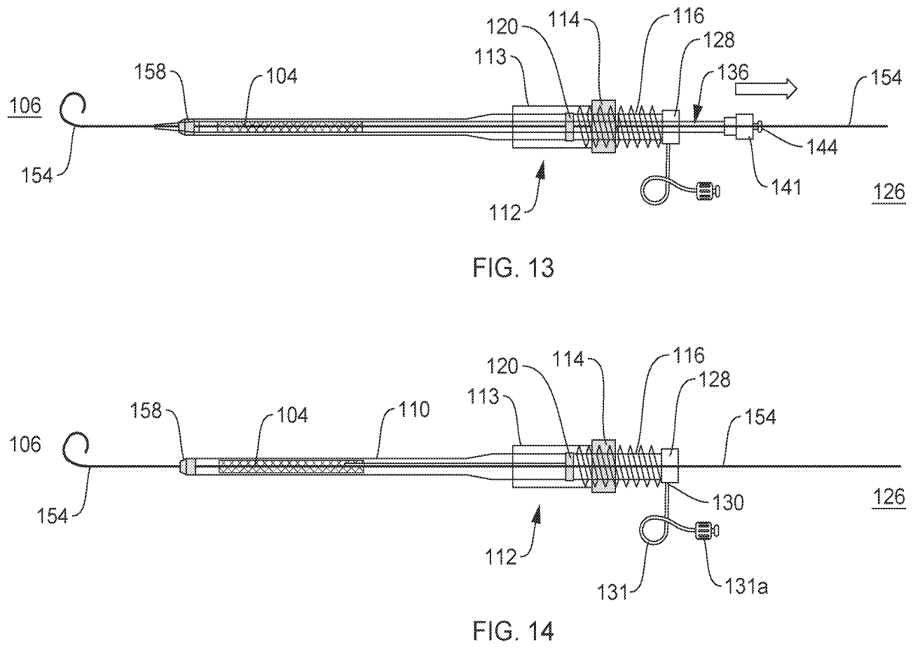

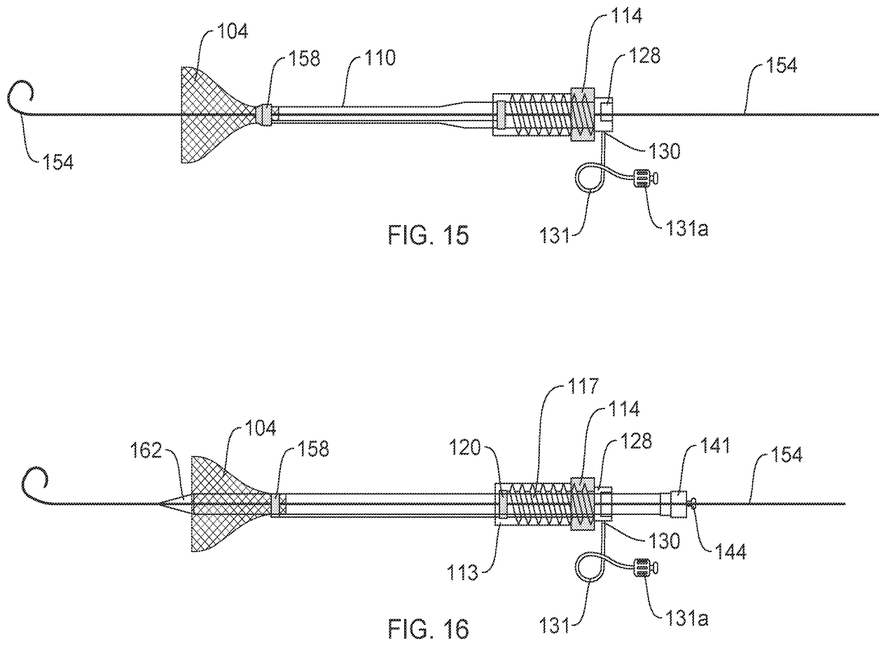

In some embodiments, some of which are illustrated in FIGS. 11-17, at least the tapered tip (and/or distal end) 158 of the expandable shaft is made of a material that can fracture or break upon an applied radial force—e.g., a force applied from within the expandable shaft and projected in a direction toward the exterior of the shaft. For example, the tapered tip (and/or distal end) of the expandable shaft can include scoring (in some embodiments, circumferential, and in some embodiments, longitudinal), which allows for such fracturing. For example, such scoring can be placed on the surface and/or within the wall of the expandable shaft via, for example, a sharp object (e.g., blade/knife), or various other manufacturing means (e.g., injection molding, 3D printing, and the like).

Accordingly, in some embodiments, the distal end (which can be a nosecone) of a dilator (or a second dilator used after a first dilator, where the first dilator is used for directing/placement of the area of the expandable shaft containing the filter) can interact with the distal end and/or distal tip of the expandable shaft to fracture or break the distal end/tip to in effect expand this area. The expansion of this area can allow for delivery of treatment devices such as larger treatment devices including, for example, prosthetic heart valves and the like.

In some embodiments of the present disclosure, an embolic protection method is provided which can be used with at least some of the EPA disclosed embodiments. Accordingly, a guide wire is inserted into the anatomy of a patient, which can be the vasculature of the patient. Over the guidewire, an EPA assembly (according to one or more embodiments of the subject disclosure) is advanced, the nosecone of the dilator (and/or the distal end or tip of the expandable shaft of the EPA assembly) along the vasculature. Once the end of the expandable shaft is positioned at or near an intended location for treatment (which can also be referred to as a treatment site), the nosecone of the dilator is undocked from the EPA assembly (e.g., disconnected or undocked from the distal and of the dilator component and/or the expandable shaft), which, in some embodiments, is done via rotation of the dilator control knob (in some embodiments, such functionality may not be needed or necessary, and thus, claims to this include a scope which would cover undocking of the nosecone via structure other than the disclosed helix and associated knob).

From there, in some embodiments, the expandable filter is advanced from the distal end of the expandable shaft/EPA (and/or dilator shaft). This, in some embodiments, can occur via rotation of the filter deployment control knob of the EPA in a corresponding first direction. In some embodiments, the length of the expandable filter can be adjusted via rotation of the filter deployment control knob in a corresponding first and/or second direction. The method may further include further (or initial) positioning the expandable filter at or near treatment site via at least one of rotation of the filter deployment control knob in the corresponding first and/or second direction, and/or movement of the expandable shaft (e.g., pushing or pulling by the operator thereof). Once the filter is deployed and properly positioned, a surgeon and/or other medical personnel can perform treatment on the patient according many treatment and therapies known to those of skill in the art. In some embodiments, this may include delivery of implants or surgical tools via the expandable shaft, which can be placed or positioned via the eyelet of the filter (and/or through the center of the filter). Accordingly, any emboli (blood emboli or other treatment debris) can be captured by the expanded filter.

After completion of embolic protection, the filter may be directed proximally back into the distal end of the expandable shaft (which can also be referred to as being recaptured. This can be done, according to some embodiments, by rotating the filter deployment control knob in the corresponding second direction so as to retract the expandable filter within the shaft (other means known to those of skill in the art may also be used). In EPA embodiments including a docking nosecone, at this point, the nosecone is directed in a proximal direction so as to dock with the end of the dilator and/or expandable shaft. In some embodiments, this can be done by rotating the dilator control knob in a corresponding second direction. Thereafter, the EPA assembly and guidewire can be removed from the anatomy of the patient.

In some embodiments, an embolic protection method is provided which can be used with one and/or another of the EPAs disclosed herein. To this end, a guidewire is inserted into the anatomy/vasculature of a patient. After which, the distal end of the expandable shaft is inserted into the vasculature of a patient, where the expandable shaft includes therein the dilator such that the nosecone of the dilator is the most distal element of the EPA. The method further includes positioning of the distal end of the EPA such that the expandable filter in the undeployed state is positioned at or adjacent an intended treatment site.

In some embodiments, the dilator is then removed from the EPA. Thereafter, and optionally, the location of the filter containing portion (i.e., the filter is in an undeployed state) of the expandable shaft can be adjusted via movement of the EPA along vasculature (by the operator of the EPA). Once location of the filter containing portion of the expandable shaft is adequately positioned, the filter is deployed. In some embodiments, the filter can be deployed by via rotation of the filter deployment knob, and, in some embodiments, a length of the deployed filter can be adjusted via rotation of the filter deployment knob (either further rotation or with initial rotation).

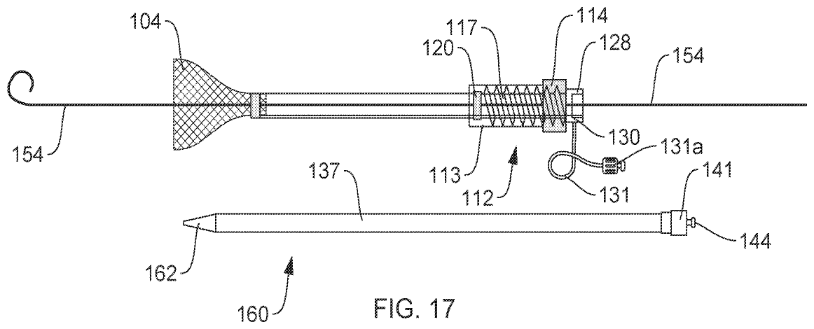

In some embodiments, the method may further and optionally include (if deemed necessary) the insertion of a second dilator 160 (see FIG. 17), which can be configured to expand the expandable shaft beyond its initial size (or a size upon which the first dilator expanded the expandable shaft to for example). The second dilator can be used, in some embodiments, to further open the distal end (and/or a tapered distal tip/end) of the expandable shaft. In some embodiments, this is done so that larger treatment devices can be delivered via the EPA (e.g., prosthetic heart valves, surgical tools, and/or the like).

In some embodiments, a treatment device can be delivered into the anatomy of the patient, which can occur through the expanded filter (i.e., within), or via the eyelet should the EPA include an eyelet, see above). After the patient is treated, and embolic protection is completed, the expandable filter may be withdrawn/recaptured by the distal end of the expandable shaft, which, in some embodiments, is performed by rotation of the filter control knob in a second direction. The EPA and guidewire can then be removed from the patient; in some embodiments, the guidewire can be removed at an earlier time, after removal of the EPA, or at the same time as removal of the EPA (this step can be used/performed/included for any of the disclosed embodiments).

Examples according to some embodiments of the disclosure:

Example 1: An embolic protection apparatus (EPA) which can include an expandable filter arranged on a distal end of the apparatus and including a plurality of pores sized to allow the flow of the blood with limited interruption and capture of emboli greater than the pore size, an expandable shaft for at least housing the expandable filter prior to deployment of the filter, a filter control wire, and a control handle including a filter deployment knob configured to move along a filter deployment helix.

Example 2: the apparatus of example 1, where the eyelet is configured to allow passage of at least one of a guide wire, dilator and one or more therapeutic devices.

Example 3: the apparatus of examples 1 or 2, where the expandable filter is connected to the filter deployment helix by at least one connector wire.

Example 4: the apparatus of any of preceding example, where a/the connector wire is comprised of at least one of stainless steel and Nitinol.

Example 5: the apparatus according to any preceding example, where the deployment helix is engaged with the filter deployment knob.

Example 6: the apparatus of any preceding example, wherein upon rotation of the filter deployment knob in a first direction, the filter is pushed out of a distal end of the expandable shaft.

Example 7: the apparatus of any preceding example, where the control handle includes a port configured to provide at least one of flushing and irrigating a component of the apparatus.

Example 8: the apparatus of example 7, wherein the component is selected from the group consisting of: the expandable shaft, guide wire, the connector wire, and the control handle.

Example 9: the apparatus of any preceding example, wherein a/the gasket is affixed to the control handle and is configured to prevent leakage from the apparatus during use.

Example 10: the apparatus of any preceding example, wherein a/the gasket prevents leakage from at least one of insertion and removal of therapeutic devices from the apparatus.

Example 11: the apparatus of any proceeding example, wherein a proximal end of the filter is connected to a distal end of a wire or cable.

Example 12: the apparatus of example 11, wherein a proximal end of the wire or cable is connected to the filter deployment helix for enabling linear movement of the expandable filter inside the expandable shaft.

Example 13: the apparatus of examples 11 or 12, wherein the wire or cable is comprised of stainless steel or nitinol.

Example 14: the apparatus of any preceding example, wherein the eyelet is configured to provide a passage for at least one of a guide wire, a dilator, and one or more therapeutic devices, through the inner surface of the filter.

Example 15: the apparatus of any preceding example, wherein the eyelet is configured to be flexible.

Example 16: the apparatus of example 15, wherein the flexibility of the eyelet is configured so as to accommodate passage therethrough of elements or devices of varying diameters.

Example 17: the apparatus of any preceding example, wherein a/the gasket is mounted on a distal end of the filter control helix and is configured to seal the proximal end of the expandable shaft.

Example 18: the apparatus of any preceding example, further comprising a first dilator.

Example 19: the apparatus of example 18, wherein a/the gasket includes an opening for receiving a distal end of the first dilator.

Example 20: the apparatus of example 18, wherein the first dilator comprises at least one of: a dilator nosecone, a dilator outer shaft, a dilator inner shaft, a dilator control handle, a nosecone control knob, a dilator helix, and a flush port.

Example 21: the apparatus of example 18, wherein the first dilator comprises at least a plurality of: a dilator nosecone; a dilator outer shaft; a dilator inner shaft; a dilator control handle; a nosecone control knob; a dilator helix; and a flush port.

Example 22: the apparatus of example 18, wherein the first dilator comprises at least a majority of: a dilator nosecone; a dilator outer shaft; a dilator inner shaft; a dilator control handle; a nosecone control knob; a dilator helix; and a flush port.

Example 23: the apparatus of example 18, wherein the first dilator comprises substantially all of: a dilator nosecone; a dilator outer shaft; a dilator inner shaft; a dilator control handle; a nosecone control knob; a dilator helix; and a flush port.

Example 24, the apparatus of example 18, wherein the first dilator comprises: a dilator nosecone; a dilator outer shaft; a dilator inner shaft; a dilator control handle; a nosecone control knob; a dilator helix; and a flush port.

Example 25: the apparatus of any of example 20-24, wherein a proximal end of at least one of the inner shaft and the outer shaft is attached to the dilator helix.

Example 26: the apparatus of any of examples 20-25, wherein a distal end of at least one of the inner shaft and the outer shaft is attached to the dilator nosecone.

Example 27: the apparatus of any of examples 20-26, wherein a proximal end of the outer shaft is attached to the control handle.

Example 28: the apparatus of any of examples 20-27, wherein a/the distal end of the outer shaft is tapered.

Example 29: the apparatus of any of examples 20-28, wherein a/the distal end of the outer shaft is configured to transition to at least one of the nosecone and the inner shaft.

Example 30: the apparatus of any of examples 20-29, where the dilator helix is engaged with the nosecone control knob.

Example 31: the apparatus of any of examples 20-30, wherein rotation of the nosecone control knob in a first direction causes the nosecone to move in a distal direction.

Example 32: the apparatus of example 31, wherein as a result of the nosecone being moved in the distal direction, the nosecone is disconnected or undocked from a distal end of outer shaft.

Example 33: the apparatus of any of examples 20-25, wherein rotation of the nosecone control knob in a second direction causes the nosecone to move in a proximal direction.

Example 34: the apparatus of example 33, wherein as a result of the nosecone moving in a proximal direction, the nosecone is connected or docked with a distal end of the outer shaft.

Example 35: the apparatus of any of examples 1-34, further comprising a radiopaque marker.

Example 36: the apparatus of example 35, wherein the radiopaque mark functions as stop for cessation of movement of the nosecone.

Example 37: the apparatus of any of examples 35-36, wherein the radiopaque marker is configured to enable verification of at least one of the movement of the nosecone and undocking and docking of the nosecone with the distal end of the outer shaft under fluoroscopy.

Example 38: the apparatus of any of examples 20-37, wherein the inner shaft includes at least one lumen.

Example 39: the apparatus of example 38, wherein the at least one lumen is configured to receive a guidewire.

Example 40: the apparatus of any of examples 20-39, wherein a/the distal end of at least one of the inner shaft and the outer shaft is comprised of a flexible material.

Example 41: the apparatus of any of examples 1-40, wherein a distal end of the expandable shaft is tapered to transition to the distal end of the first dilator.

Example 42: the apparatus of example 41, wherein the tapered distal end of the shaft is comprised of a material capable of being fractured or broken under an outward radial force.

Example 43: the apparatus of examples 41 or 42, wherein the tapered distal end of the expandable shaft includes longitudinal scoring.

Example 44: the apparatus of example 43, wherein the scoring is configured to enable controlled fracturing or breaking of the distal end.

Example 45: the apparatus according to any of examples 18-44, further comprising a second dilator having a diameter greater than a diameter of the first dilator.

Example 46: the apparatus of example 45, wherein the second dilator comprises at least one of: a second dilator nosecone; a second dilator outer shaft; a second dilator inner shaft; a second dilator control handle; a second nosecone control knob; a second dilator helix; and a second flush port.

Example 47: the apparatus of example 45, wherein the first dilator comprises at least a plurality of: a second dilator nosecone; a second dilator outer shaft; a second dilator inner shaft; a second dilator control handle; a second nosecone control knob; a second dilator helix; and a second flush port.

Example 48: the apparatus of example 45, wherein the first dilator comprises at least a majority of: a second dilator nosecone; a second dilator outer shaft; a second dilator inner shaft; a second dilator control handle; a second nosecone control knob; a second dilator helix; and a second flush port.

Example 49: the apparatus of example 45, wherein the first dilator comprises substantially all of: a second dilator nosecone; a second dilator outer shaft; a second dilator inner shaft; a second dilator control handle; a second nosecone control knob; a second dilator helix; and a second flush port.

Example 50: the apparatus of example 45, wherein the first dilator includes: a second dilator nosecone; a second dilator outer shaft; a second dilator inner shaft; a second dilator control handle; a second nosecone control knob; a second dilator helix; and a second flush port.

Example 51: an embolic protection apparatus (EPA) including: an expandable filter arranged on a distal end of the apparatus and including a plurality of pores sized to allow the flow of the blood with limited interruption and capture of emboli greater than the pore size, an expandable shaft for at least housing the expandable filter prior to deployment of the filter; a filter control wire; a control handle including a filter deployment knob configured to move along a filter deployment helix; and a dilator including a dilator nosecone, a dilator outer shaft, a dilator inner shaft, a dilator control handle, a nosecone control knob, a dilator helix.

Example 52: an embolic protection apparatus (EPA) including: an expandable filter arranged on a distal end of the apparatus and including a plurality of pores sized to allow the flow of the blood with limited interruption and capture of emboli greater than the pore size; an expandable shaft for at least housing the expandable filter prior to deployment of the filter; a filter control wire; a control handle including a filter deployment knob configured to move along a filter deployment helix; a first dilator including a dilator nosecone, and a dilator outer shaft; and a second dilator including a second dilator nosecone and a second dilator outer shaft.

Example 53: an embolic protection apparatus comprising a dilator including a shaft, a distal tapered tip, a lumen for receiving a guidewire, and a luer-lock connector arranged at a proximal end thereof.

Example 54: an embolic protection method including: providing (optionally) an embolic protection apparatus (EPA) according to any one or more of examples 1-44 and 51; inserting a guide wire into an anatomy of a patient; advancing the expandable shaft of the EPA containing the expandable filter of the EPA over the guide wire; undocking the nosecone from a distal end of the shaft of the EPA via rotation of a dilator control knob in a corresponding first direction; advancing the expandable filter from the distal end of the EPA and dilator shaft via rotation of the filter deployment control knob of the EPA in a corresponding first direction, wherein a length of the expandable filter is adjusted via rotation of the filter deployment control knob in a corresponding first and/or second direction; positioning the expandable filter in the intended treatment site via at least one of rotation of the filter deployment control knob in the corresponding first and/or second direction, and movement of the expandable shaft; after completion of embolic protection: rotating the filter deployment control knob in the corresponding second direction so as to retract the expandable filter within the shaft; and rotating the dilator control knob in a corresponding second direction so as to dock a distal end of the dilator control knob with the distal end of the dilator shaft. The method of the example may further include removing the EPA from the anatomy.

Example 55: the method of example 54, wherein the dilator is removed from the anatomy while the expandable filter is deployed.

Example 56: an embolic protection method including: providing (optionally) an embolic protection apparatus (EPA) according to any one or more of examples 45-50 and 52; inserting the distal end of the expandable shaft into the vasculature of a patient, the expandable shaft having therein the dilator such that the nosecone of the dilator is the most distal element of the EPA; positioning the distal end of the EPA such that the expandable filter in the undeployed state is positioned at or adjacent an intended treatment site; removing the dilator from the EPA; adjusting the location of the filter in an undeployed state, if necessary, via movement of the EPA along vasculature; deploying the filter by via rotation of the filter deployment knob, wherein a length of the deployed filter can be adjusted via rotation of the filter deployment knob; inserting the second dilator, the second dilator configured to expand the expandable shaft and open the tapered distal end of the expandable shaft; delivering a treatment device into the anatomy of the patient, the delivery occurring through the expanded filter; withdrawing or recapturing the expandable filter by rotation of the filter control knob in a second direction; and withdrawing the EPA from the patient.

### General Matters

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that any and all parameters, dimensions, materials, and configurations described herein are meant to be an example and that the actual parameters, dimensions, materials, and/or configurations will depend upon the specific application or applications for which the inventive teachings disclosed herein is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the claims supported by the disclosure, and equivalents thereto, inventive embodiments may be practiced otherwise than as specifically described and claimed. Inventive embodiments of the present disclosure are also directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Embodiments disclosed herein may also be combined with one or more features, as well as complete systems, devices and/or methods, to yield yet other embodiments and inventions. Moreover, some embodiments, may be distinguishable from the prior art by specifically lacking one and/or another feature disclosed in the particular prior art reference(s); i.e., claims to such embodiments are distinguishable from the prior art by including one or more negative limitations.

Also, various inventive concepts may be embodied as one or more methods, of which examples has been provided. The acts performed as part of the method may be ordered in any suitable way. Accordingly, embodiments may be constructed in which acts are performed in an order different than illustrated, which may include performing some acts simultaneously.

The indefinite articles “a” and “an,” as used herein in the specification and in the claims, unless clearly indicated to the contrary, should be understood to mean “at least one.” The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. (canceled)

2. A method for embolic protection, said method comprising:
(a) inserting a guide wire into a subject;
(b) advancing an expandable shaft of an embolic protection apparatus over the guide wire,
wherein the embolic protection apparatus comprises (i) an expandable filter coupled to a distal end of the embolic protection apparatus, wherein the expandable filter comprises a plurality of pores configured to capture emboli greater than a pore size of the plurality of pores, (ii) the expandable shaft which is configured to house the expandable filter, and (iii) a control handle comprising a filter deployment control knob configured to move along a filter deployment helix;
(c) advancing the expandable filter from the distal end of the embolic protection apparatus via rotation of the filter deployment control knob in a first direction; and
(d) positioning the expandable filter to a treatment site in the subject via the rotation of the filter deployment control knob in the first direction and/or a second direction.

3. The method of claim 2, further comprising after (b), undocking a nosecone from a distal end of the expandable shaft via rotation of a dilator control knob of a dilator in a first direction.

4. The method of claim 3, further comprising rotating the dilator control knob in the second direction to dock a distal end of the dilator control knob within the distal end of the dilator shaft.

5. The method of claim 3, further comprising inserting the distal end of the expandable shaft into a vasculature of the subject, wherein the expandable shaft is configured such that the nosecone of the dilator is the most distal element of the embolic protection apparatus.

6. The method of claim 5, further comprising positioning the distal end of the embolic protection apparatus such that the expandable filter in an undeployed state is positioned at or adjacent the treatment site.

7. The method of claim 6, further comprising removing the dilator from the embolic protection apparatus and, optionally, adjusting a location of the expandable filter in the undeployed state by moving the embolic protection apparatus along the vasculature.

8. The method of claim 7, further comprising deploying the expandable filter by rotating the filter deployment control knob, wherein a length of a deployed expandable filter can be adjusted by rotating the filter deployment control knob.

9. The method of claim 8, further comprising inserting a second dilator, wherein the second dilator is configured to expand the expandable shaft and open a tapered distal end of the expandable shaft.

10. The method of claim 9, further comprising delivering a treatment device through the expandable filter into the subject.

11. The method of claim 10, further comprising withdrawing or recapturing the expandable filter by rotating the filter deployment control knob in the second direction.

12. The method of claim 11, further comprising withdrawing the embolic protection apparatus from the subject.

13. The method of claim 2, in (c), wherein a length of the expandable filter is adjusted via rotation of the filter deployment control knob in the first direction and/or a second direction.

14. The method of claim 2, further comprising rotating the filter deployment control knob in the second direction to retract the expandable filter within the expandable shaft.

15. The method of claim 2, further comprising removing the embolic protection apparatus from the subject.

16. The method of claim 2, wherein the control handle comprises a port configured to flush a component of the embolic protection apparatus.

17. The method of claim 16, wherein the component is selected from the group consisting of: the expandable shaft, the guide wire, a connector wire, and the control handle.

18. The method of claim 2, in (b), wherein the expandable shaft of the embolic protection apparatus is advanced over the guide wire during delivery or implantation of a prosthetic heart valve into the heart of the subject.

19. The method of claim 2, in (b), wherein the expandable shaft of the embolic protection apparatus is advanced over the guide wire during a cardiac or blood vessel procedure.

20. The method of claim 2, in (b), wherein the expandable shaft of the embolic protection apparatus is advanced over the guide wire during a Transcatheter Aortic Valve Replacement (TAVR) procedure.

21. The method of claim 2, in (b), wherein the expandable shaft of the embolic protection apparatus is advanced over the guide wire during a Transcatheter Mitral Valve Replacement (TMVR) procedure.
